(19) United States
(12) Patent Application Publication
(10) Pub. No.: US 2024/0206525 A1
(43) Pub. Date: Jun. 27, 2024

(54) METHOD FOR DETERMINING AN ADDITIVE CONTENT IN A TOBACCO PAPER FOR ELECTRIC CIGARETTES

(71) Applicant: TEWS Electronik GmbH & Co. KG, Hamburg (DE)

(72) Inventors: Udo Schlemm, Hamburg (DE); Hendrik Richter, Hamburg (DE)

(73) Assignee: TEWS Electronik GmbH & Co. KG, Hamburg (DE)

(21) Appl. No.: 18/288,142

(22) PCT Filed: Apr. 26, 2022
(86) PCT No.: PCT/EP2022/061054

§ 371 Date: Oct. 24, 2023

(30) Foreign Application Priority Data
Apr. 27, 2021 (DE) 10 2021 110 760.1

(51) Int. Cl. A24B 15/28; A24B 9/00; A24B 15/22; A24C 5/34

(52) U.S. Cl. CPC A24B 15/282; A24B 9/00; A24B 15/22; A24C 5/3412

(57) ABSTRACT

A method is provided for measuring an additive content in a tobacco paper for electric cigarettes produced from a pulpy mass of additives, water, flavoring substances and tobacco that is dried to form a tobacco paper having a single-layer. The method includes measuring the tobacco paper using at least one microwave resonator having two resonance modes with two resonant frequencies (f_{L}, f_{H}). A lower of the two resonant frequencies (f_{L}) is in a range that is less than 1 GHz and a higher of the two resonant frequencies is in a range that is more than 2 GHZ. A density-independent moisture value (Φ_{L,H}) is determined for each of the two resonance modes and a glycerol content (g) is determined depending on two moisture angles.

### CROSS REFERENCE TO RELATED INVENTION

This application is a national stage application pursuant to 35 U.S.C. § 371 of International Application No. PCT/EP2022/061054, filed on Apr. 26, 2022, which claims priority to, and benefit of, German Patent Application No. 10 2021 110 760.1, filed Apr. 27, 2021, the entire contents of which are hereby incorporated by reference.

### TECHNOLOGICAL FIELD

The present disclosure relates to a method for measuring an additive content in a tobacco paper for electric cigarettes.

### BACKGROUND

Glycerol (E422) is an additive which is used as a humectant for tobacco products. In cigarette and pipe tobacco, the humectant is intended, above all, to extend the storage time of the product and to prevent it from drying out. In shisha tobacco, higher amounts of humectant are added to the tobacco in order to prevent combustion of the tobacco on the one hand and to generate a vapor that is as dense as possible on the other hand. Furthermore, glycerol is used as a fog fluid in electric cigarettes, where a dense white vapor forms under the influence of heat.

During the production of a particular type of electric cigarettes, glycerol, binder, flavoring substances and tobacco as well as further aerosol-forming additives are mixed to form a pulp. Said pulp is rolled and dried, creating a so-called tobacco paper. Said tobacco paper is then crimped, for example processed into a rod in a crimper. During production of the pulp, a defined proportion of glycerol is added. However, an undefined proportion hereof can be lost during the drying process. A subsequent check of the glycerol content is therefore very important for ensuring the quality of the final product.

DE 10 2007 041 429 A1 discloses a method for measuring a moisture value F of dielectric substances using at least one microwave resonator, wherein a shift in the resonant frequency A is evaluated in each case for at least two resonance modes each having different resonant frequencies from one another and a density-independent moisture value is calculated from the measured shifts in the resonant frequency. It is known to measure the resonant frequency shifts for widely separated resonant frequencies. In the process, a resonant frequency of less than 1 GHz is measured, whereas the other resonant frequency shift is measured at a frequency of more than 7 GHz.

WO 2017/080982 A1 discloses a device and a method for determining the proportion of at least one additive in a tobacco-containing substance. In order to determine the proportions of tobacco and water, two measurands are obtained from an alternating electromagnetic field. It is further explained that, in order to determine the proportion of at least one further additive, a further measurand must be obtained by measurement with a second alternating electromagnetic field in the case of a second measuring frequency. As the measurands, for example the magnitude and phase of the alternating electromagnetic fields or resonance shift and resonance broadening, are measured independently of one another at two different measuring frequencies, a total of four measurands are available from which conclusions can be drawn about the proportions by weight of tobacco, water and the at least one additive. It is further noted that three measurands are fundamentally sufficient for determining tobacco, water and an additive, but four measurands increase the accuracy of the measurement further. This approach known from the prior art consists in advantageously determining the moisture content and the proportions by weight of the tobacco and of the additive from the at least four determined measurands in a data processing device as the best solution of an overdetermined system of equations, for example with minimum error squares. The proportions by weight of additives can be determined in addition to the moisture content and proportion by weight of the tobacco.

### BRIEF SUMMARY OF THE INVENTION

The object of the invention is therefore to provide a method for measuring the additive content in a tobacco paper which delivers the most accurate values possible using simple means.

An embodiment of the disclosed method for measuring the additive content in a tobacco paper for electric cigarettes as accurately as possible is provided. The additive content is determined in vol. % or wt. %. The tobacco paper is produced from a pulpy mass of additives, water, flavoring substances and tobacco, wherein the pulpy mass is preferably dried after a rolling step to form a single-layer tobacco paper. During the drying process, an unpredictable amount of additives and water escapes, and therefore a measurement must take place at the tobacco paper in order to determine the additive content. The measurement takes place using at least one microwave resonator having two resonance modes with two different resonant frequencies. The lower of the two resonant frequencies is in a frequency range of less than 1 GHz, and the higher of the two resonant frequencies has values of more than 2 GHZ, wherein the lower range may be in a lower microwave range of 800 MHz. A density-independent moisture value is calculated for each of the two resonant frequencies, wherein the additive content is determined depending on the two density-independent moisture values. The density-independent moisture value is preferably in each case a density-independent moisture angle. The respective density-independent moisture value is characterized in that it is independent of the density and is indicative of a moisture content in the measured material. Unlike in the prior art, four measured values are not combined into a system of equations, but rather the values acquired by means of the two resonance modes are processed into density-independent moisture values. As the additive content is determined independently of the tobacco content via a density-independent variable such as the moisture angle, significantly more accurate values can be achieved. In physics terms, this means that the evaluation of state variables that present themselves as intensive state variables provides significantly better results than the evaluation of extensive state variables.

Preferably, glycerol is used as an additive in the tobacco-processing industry. Especially for glycerol, the density-independent moisture angles provide very accurate results.

In a preferred embodiment, the glycerol content g is determined linearly from both moisture angles and an offset value. It is important for the glycerol content g that both moisture angles, i.e. of the resonant frequency from the high-frequency range as well as of the resonant frequency in the microwave range, contribute to the glycerol content.

In another preferred embodiment, a moisture content is measured for the tobacco paper, depending on the moisture value for the higher frequency. It is important to recognize that the measurement of the moisture content of the tobacco paper only depends on the density-independent moisture value for the higher frequency and any contributions from the lower resonant frequency can be neglected. The measurement of the glycerol content in particular does not show this behavior, since it depends on both moisture angles.

In a preferred embodiment, the at least one microwave resonator is configured as a planar sensor. The planar sensor has a field that exits the resonator body and that interacts with the measured object. When using a planar sensor, the tobacco paper is filtered over a flat sensor surface and thereby transported through a measuring field. In addition to planar sensors, gap sensors may in principle also be provided, in which the tobacco paper is transported through a resonator cavity via a gap.

In an embodiment, the measurement can take place directly at the single-layer tobacco paper. However, it has been shown that the measurement can also take place at the tobacco paper that has been rolled up into a bobbin. In principle, it is also possible to perform both measurements one after the other. Alternatively or additionally, the measurement can also take place during or immediately after the drying step. This measurement can take place after the drying process is complete or at a defined point in time during the drying process.

In a preferred embodiment, it is possible to measure the glycerol content of the tobacco paper prior to same being processed further into a rod, i.e. before it enters the crimper.

The disclosed measuring method is very reliable and can control the addition of water and/or glycerol to the pulpy mass depending on the measured moisture angle. In this way, a desired value for the glycerol and moisture content can be adjusted.

In a preferred embodiment, the moisture value is a moisture angle. The moisture angle is determined as the quotient of the resonant frequency shift and the broadening of the full width at half maximum. During the resonant frequency shift, the changes in frequency in Hertz between an empty and filled resonator are compared with one another. The full width at half maximum of the resonance curve with an unfilled resonator is also taken into account. Instead of the full width at half maximum, it is also possible to take into account other variables caused by the damping of the resonance, for example the amplitude of the resonance curve. It has also proven advantageous to determine the moisture angle as the arc tangent of the density-independent quotient of the resonant frequency shift and the broadening of the full width at half maximum.

### BRIEF DESCRIPTION OF THE DRAWINGS

The method according to the invention will be further explained below with reference to an exemplary embodiment.

FIG. 1 schematically depicts locations for glycerol measurement in the primary.

FIG. 2 schematically depicts possible locations for the glycerol measurement in the crimper.

FIG. 3 illustrates the measured values for the moisture angle of two modes depending on the moisture and glycerol content.

FIG. 4 graphically illustrates results of the glycerol measurement.

### DETAILED DESCRIPTION OF THE INVENTION

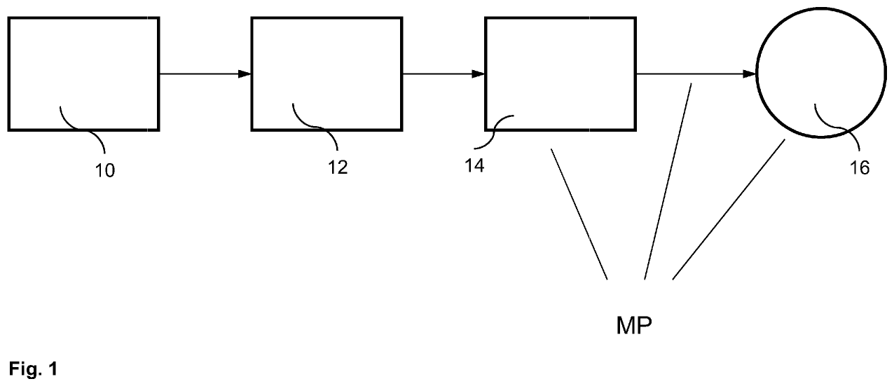

FIG. 1 schematically illustrates a mixer 10, in which glycerol is mixed with water, binder, flavoring substances, further aerosol-forming additives and tobacco to form a pulp for use in electric cigarettes. The exemplary embodiment is based on glycerol, but other additives with or without glycerol may also be used and measured in the same way. Here, the ingredients are added in precisely defined ratios and processed in the mixer into a homogeneous mixture in the form of a pulp. The pulp formed in this manner is rolled in a rolling machine 12 and fed to the dryer 14 as a flat material. The drying process takes place in the dryer 14, during which an undefined amount of water and glycerol is lost from the tobacco paper. The dried tobacco paper is rolled up into a bobbin in 16. Possible measuring stations MP for monitoring the moisture content and measuring the glycerol content are located, for example, in the dryer 14, along the route of the dried tobacco paper from the dryer 14 to the bobbin 16 and directly at the bobbin 16. The values obtained for the glycerol content and/or moisture content can be fed back to the mixer 10 in order to adjust the glycerol content to a desired value. It is also possible to adjust the moisture content. The measured values can also be used to adapt the parameters of the drying process to the desired values for moisture and glycerol.

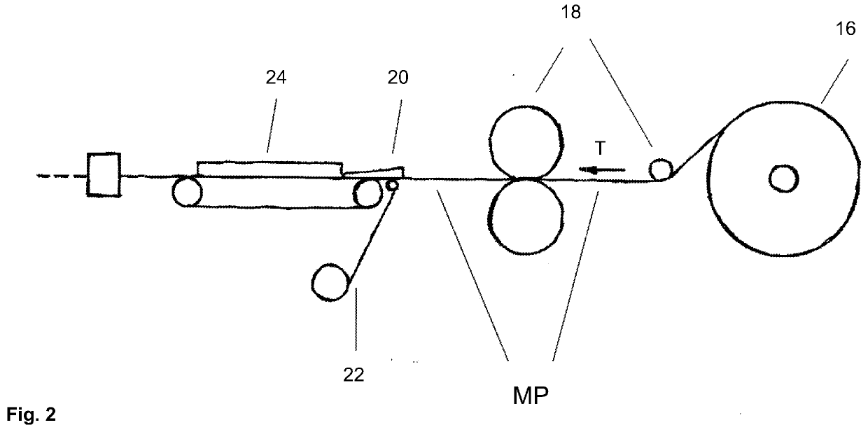

FIG. 2 shows, in a schematic view, how tobacco paper 18 is unwound from the bobbin 16 in the transport direction T. The tobacco paper is fed to a crimper 20 via transport rollers 18. Paper 22 for rod wrapping is also fed to the crimper 20 in order to be formed into a rod in the region 24. The possible measuring stations MP are located along the route from the bobbin to the crimper 20, to which further paper is fed for forming a rod.

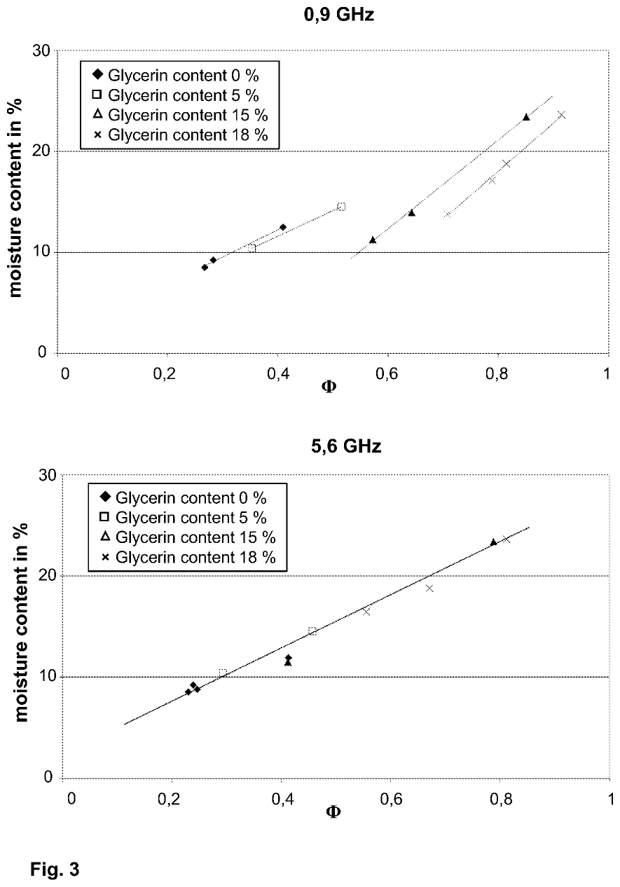

FIG. 3 shows the plotted moisture values (moisture content in %) over the moisture angle Φ for two frequencies with 0.9 GHZ and 5.6 GHz. The moisture angle Φ is formed as the arc tangent of the quotient of B/A, wherein A describes the resonant frequency shift and B describes the broadening of the resonance curve. The measurements were performed at a different glycerol content and different moisture content. The second measurement was taken at the same material for the higher frequency.

The measurement at 5.6 GHz shows that the moisture angles Φ are independent of the glycerol content of the samples and only depend on the moisture content thereof. This results from the proportionality of the moisture angle and moisture content at a different glycerol content. Therefore, the regression line plotted in FIG. 3 can be used as a calibration for a glycerol-independent moisture measurement.

In contrast, the measurement at 0.9 GHz shows that the measured moisture angles Φ depend on the moisture and glycerol content. The samples with the same glycerol content are denoted in the figure by separate linear regressions. To compensate for the influence of the variation in the material moisture content, the moisture angles Φ of both frequencies must be taken into account for the measurement of the glycerol content.

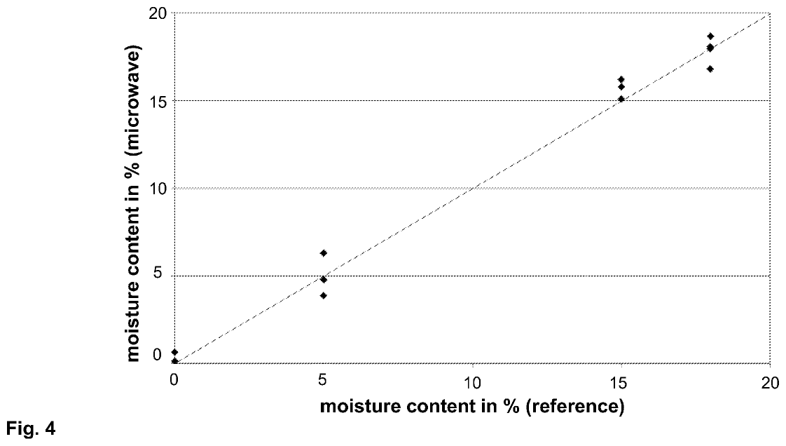

FIG. 4 shows the result of the evaluation, wherein the reference value for the glycerol content has been plotted against the measured glycerol value. The calculation takes place using the moisture angles Φ of both frequencies in the calibration equation explained below. The good match between the values and the regression line can be clearly seen, with the measured values deviating by only a few percent from the reference values.

The measured value of the resonance mode with the high frequency PH is used for the measurement of the moisture content. One approach for the moisture value u is as follows:

$$u = {a}_{1} \cdot {Φ}_{H} + {a}_{2}$$

wherein a₁, a₂ represent calibration coefficients here. If the calibration coefficients are determined, the moisture value can be determined directly from the measured moisture angle Φ_{H}.

The moisture angle of both modes is used to determine the glycerol content:

$$g = {b}_{1} \cdot {Φ}_{L} + {b}_{2} \cdot {Φ}_{H} + {b}_{3}$$

wherein b₁, b₂ and b₃ are the calibration coefficients. It is important here that both moisture angles are included in the determination of the glycerol content as a mass-independent variable and thus the measurement of moisture and glycerol content is independent of the mass of the measured product. The mass fraction of the measured product, determined as in the prior art via a possibly overdetermined system of equations, impairs the measurement accuracy. The additional determination of the proportion of tobacco cannot take place in the approach according to the invention, which is based on mass-independent measurands.

1-14. (canceled)

15. A method for measuring an additive content in a tobacco paper for electric cigarettes produced from a pulpy mass of additives, water, flavoring substances and tobacco that is dried to form a tobacco paper having a single-layer, comprising:
measuring the tobacco paper using at least one microwave resonator having two resonance modes with two resonant frequencies (f_{L}, f_{H}), wherein a lower of the two resonant frequencies (f_{L}) is in a range that is less than 1 GHz and a higher of the two resonant frequencies is in a range that is more than 2 GHZ;
determining a density-independent moisture value (Φ_{L,H}) for each of the two resonance modes; and
determining a glycerol content (g) depending on two moisture angles.

16. The method according to claim 15, wherein the additives at least partially comprises glycerol.

17. The method according to claim 15, wherein a content (g) of the additives depends linearly on both density-independent moisture values (Φ_{L,H}) and an offset value.

18. The method according to any one of claim 17, further comprising measuring a moisture content for the tobacco paper depending on the density-independent moisture value (Φ_{L,H}) at the higher resonant frequency.

19. The method according to claim 17, comprising determining the content (g) of the additives independently of a mass of the tobacco paper.

20. The method according to claim 15, wherein the at least one microwave resonator comprises a planar sensor.

21. The method according to claim 15, wherein the at least one microwave resonator comprises a gap sensor.

22. The method according to claim 15, wherein the measuring the tobacco paper takes place at the single-layer.

23. The method according to claim 15, wherein the measuring the tobacco paper takes place at a point where the tobacco paper has been wound up into a bobbin.

24. The method according to claim 15, wherein the measuring the tobacco paper takes place in or immediately downstream of a dryer.

25. The method according to claim 15, wherein the measuring the tobacco paper takes place upstream of a crimping apparatus.

26. The method according to claim 15, further comprising adding an amount of at least one of water and glycerol to the pulpy mass takes place depending on at least one density-independent moisture value.

27. The method according to claim 15, further comprising:
providing a moisture angle (Φ) as the moisture value; and
determining the moisture angle as a quotient of a broadening of a full width at half maximum (B) and a resonant frequency shift (A), wherein an empty and a filled resonator are compared with one another in each case.

28. The method according to claim 15, further comprising providing a moisture angle (Φ) as the moisture value, wherein the moisture angle results as an arc tangent of a quotient of a broadening of a full width at half maximum (B) and a resonant frequency shift (A).
